(19) United States
(12) Patent Application Publication
(10) Pub. No.: US 2023/0301743 A1
(43) Pub. Date: Sep. 28, 2023

(54) METHOD FOR IDENTIFYING A SITE FOR SURGICAL REMOVAL

(71) Applicant: Devicor Medical Products, Inc., Cincinnati, OH (US)

(72) Inventors: John S. Fisher, Belleair, FL (US); Frederick A. Ahari, Belleair Beach, FL (US)

(21) Appl. No.: 18/203,846

(22) Filed: May 31, 2023

Related U.S. Application Data
(63) Continuation of application No. 16/173,717, filed on Oct. 29, 2018.
Application No. 16/173,717 is a continuation of application No. 12/969,706, filed on Dec. 16, 2010, now abandoned.

(51) Int. Cl. A61B 90/00; A61B 17/34

(52) U.S. Cl. CPC A61B 90/39; A61B 17/3403; A61B 2090/3983; A61B 2090/3908; A61B 2090/3966; A61B 2090/3937; A61B 2090/3987; A61B 2010/045

(57) ABSTRACT

A method for marking a biopsy site after a biopsy has been performed includes the steps of placing a marker in the form of a semi-permeable membrane at the biopsy site so that the marker can be found at a later time, inserting a needle into the marker at the later time which is preferably immediately before a planned surgical resection of the biopsy site, and injecting a dye directly into the marker to color the marker. The semi-permeable qualities of the marker facilitate the slow egress of dye into tissue that is immediately adjacent the marker, enlarging the footprint of the marker. The semi-permeable membrane includes a high percentage of water after reaching osmotic equilibrium, rendering the marker highly visible under ultrasound imaging. The semi-permeable membrane may take the form of a fully or partially dehydrated hydrogel.

### BACKGROUND OF THE INVENTION

### 1. Field of the Invention

This invention relates, generally, to biopsy procedures. More particularly, it relates to a method for enhancing the visibility of a marker that is used to identify the location of a biopsy.

### 2. Description of the Prior Art

A month or more may elapse from the time a biopsy is completed to the time when surgery, if needed, is performed. It is therefore conventional practice to leave a marker at a biopsy site at the conclusion of a biopsy procedure so that the site can be found at a later time with the use of various imaging means.

The present inventor has previously disclosed the use of fully or partially dehydrated hydrogels as suitable markers. As the hydrogel becomes hydrated, it expands in size and can be found long thereafter by conventional imaging means.

The hydrogel marker can be found with conventional imaging means but it would represent a significant advance in the art if it could be improved by making it even easier to locate.

Dyes have been injected into tissue in an effort to mark the location of a biopsy site but dyes have a high viscosity and therefore they diffuse quickly and widely over a large area. Therefore they have little or no utility as marking agents.

Thus there is a need to provide an improved method for locating markers that harnesses the high visibility of dyes but which is not subject to the well-known disadvantages of dyes.

However, in view of the art considered as a whole at the time the present invention was made, it was not obvious to those of ordinary skill in the art that such a need existed and thus it was not obvious how the need could be met.

### SUMMARY OF THE INVENTION

The long-standing but heretofore unfulfilled need for an improved method for finding a biopsy site after a biopsy has been performed is now met by a new, useful, and non-obvious invention.

The novel method for marking a biopsy site after a biopsy has been performed includes the steps of placing a marker in the form of a semi-permeable membrane at the biopsy site so that the marker can be found at a later time, inserting a needle into the marker and injecting a dye directly into the marker with a needle to color the marker at the time the marker is placed into position. The dye does not diffuse quickly and widely throughout adjacent tissue because the semi-permeable membrane prevents immediate leakage of the dye. Instead, a small amount of dye may seep slowly through the semi-permeable membrane, thereby advantageously slightly increasing the visible footprint of the marker. The small amount of dye that seeps through the semi-permeable membrane will not diffuse widely throughout adjacent tissue because it will remain linked to the larger body of dye within the semi-permeable membrane as the semi-permeable membrane seeks osmotic balance with the liquids in the tissue within which the semi-permeable membrane is embedded.

In a second embodiment, no dye is injected at the time the marker is initially positioned and the marker is found at a later time by conventional imaging means. The needle is then inserted into the marker, preferably just prior to a surgical resection of the biopsy site, and a dye is injected directly into the marker with a needle to color the marker to thereby facilitate identification of the marker during said surgical resection.

The semi-permeable qualities of the marker facilitate the slow egress of dye from the marker into tissue that immediately surrounds the marker, thereby identifying the tissue immediately adjacent the marker and thus further identifying the biopsy site by increasing the footprint of the marker.

The steps may also include the step of fully dehydrating the semi-permeable membrane before placing it at the biopsy site, or partially dehydrating the semi-permeable membrane before placing it at the biopsy site.

The semi-permeable membrane is preferably provided in the form of a hydrogel.

The marker is found at said later time by using imaging guidance means such as ultrasound, CT, MRI, PET, fluoroscopic imaging, and the like.

The dye is selected from a group of medical-grade dyes including methylene blue, tolulene blue, and the like.

In addition to injecting said medical-grade dyes, various therapeutic agents may also be injected into the marker with the dye to provide a high concentration of said therapeutic agents in a very localized area. The therapeutic agents may be provided in the form of chemotherapy drugs, antibiotics, radioactive materials, and the like. The dye could also contain a radioopaque liquid such as iodinated contrast that would render it visible under x-ray/mammographic imaging.

The dye, the therapeutic agents, the radioopaque liquid, or any combination thereof, may instead be injected into tissue immediately adjacent the marker. The marker will then absorb the dye, therapeutic agents, radioopaque liquid, or said combination, by the process of osmosis.

In another, a contrast agent such as a radioopaque liquid, MRI contrast agent or radioactive substance is injected into the marker instead of a dye. A mammogram is then used to locate the biopsy site in a conventional way. The dye, or a dye and a therapeutic agent, can then be injected into the marker, or into tissue that is immediately adjacent to the marker, just prior to surgical resection of the biopsy site.

A localization wire of the type typically used when a mammogram is employed may be advanced into the marker to further enhance identification of the biopsy site. The dye or therapeutic agent, or both, can be injected into the marker through the needle portion of a localization wire system followed by deployment of the wire through the marker by advancing the wire through the needle.

The primary object of this invention is to enhance the ability to locate a marker that is previously positioned at a biopsy site so that the biopsy site may be more easily found at a later time during surgical resection of the biopsy site. Compounds having utility as marking agents include, for example, fludeoxyglucose (¹⁸F) or fluorodeoxyglucose (¹⁸F), commonly abbreviated ¹⁸F-FDG or FDG. This enables detection by a radioactive detector device.

A second compound is a fluorescent material including nano particles, such as plasmonic fluorescent quantum dots, which are injected into the hydrogel to facilitate locating tumor cells under different wavelength light sources, such as UV light, during surgery. Liposome-based nanocapsules may also be used as marking agents.

A more particular object is to facilitate the location of such a marker by the use of medical-grade dyes that are injected directly into the marker or into tissue immediately surrounding the marker.

Another important object is to disclose the use of a fully or partially hydrated semi-permeable membrane, such as a hydrogel, as said marker.

These and other important objects, advantages, and features of the invention will become clear as this description proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the nature and objects of the invention, reference should be made to the following detailed disclosure, taken in connection with the accompanying drawings, in which:

FIG. 1 depicts a semi-permeable membrane when in its fully dehydrated condition and prior to the introduction of any dye thereinto;

FIG. 2 depicts the semi-permeable membrane of FIG. 1 when in a hydrated condition and after a dye has been injected thereinto; and

FIG. 3 depicts the semi-permeable membrane in a hydrated condition after dye has been injected thereinto where the semi-permeable membrane has been in a patient's body for a time sufficient for some of the dye to have spread from the semi-permeable membrane into surrounding tissue of the patient's body.

### DETAILED DESCRIPTION OF THE PREFERRED EMBODIMENT

The novel method for marking a biopsy site after a biopsy has been performed includes the step of placing a marker in the form of a semi-permeable membrane in a fully dehydrated state at the biopsy site so that the marker can be found at a later time under imaging guidance means such as ultrasound, CT, X-ray, MRI, PET, fluoroscopic imaging, radiation emitting compounds that are detected with a gamma camera, and the like.

A partially dehydrated semi-permeable membrane may also be used instead of a fully dehydrated semi-permeable membrane.

The preferred embodiment of this invention employs a hydrogel as the preferred semi-permeable membrane. The semi-permeable membrane includes a high percentage of water after reaching osmotic equilibrium, rendering the marker highly visible under ultrasound imaging.

In a first embodiment, a needle is inserted into the marker and a medical dye is injected into the marker at the time of the biopsy procedure.

In a second embodiment, no medical dye is inserted into the marker at the time of the biopsy procedure and the marker is found at a later date using the aforesaid conventional imaging guidance means. The dye is then injected into the marker immediately prior to a surgical resection.

The needle is used in both embodiments to inject a medical dye such as methylene blue, toluene blue, gentian violet, methyl violet, dichlorophenolindophenol, fluorescein, Prussian blue, Egyptian blue, Han purple, potassium ferrocyanide, potassium ferricyanide, phenothiazine, or the like directly into the marker. The dye colors the marker to facilitate identification of the marker at the time of a surgical removal of a lesion at said biopsy site.

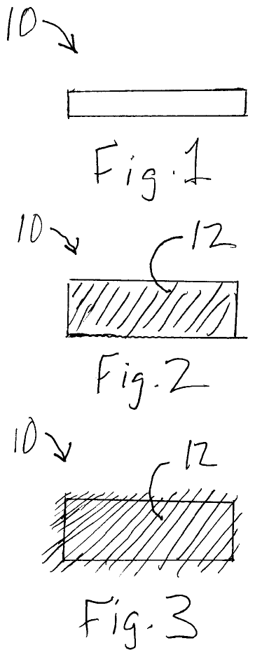

FIG. 1 depicts semi-permeable membrane 10 when in its fully dehydrated condition and prior to the introduction of any dye thereinto.

FIG. 2 depicts semi-permeable membrane 10 when in a hydrated condition and after dye 12, represented by cross-hatching, has been injected thereinto.

FIG. 3 depicts semi-permeable membrane 10 in a hydrated condition after dye 12 has been injected thereinto and where semi-permeable membrane 10 has been in a patient's body for a time sufficient for some of dye 12 to have spread from semi-permeable membrane 10 into surrounding tissue of the patient's body.

The semi-permeable quality of the marker facilitates the egress of dye into the immediate surrounding tissue, identifying not just the marker but also the tissue immediately adjacent the marker, thereby enlarging the footprint of the marker and thus facilitating identification of the biopsy site.

The marker may also be injected with therapeutic agents including chemotherapy drugs, antibiotics and radioactive material for the purpose of high drug, antibiotic, and radiation concentration in a very localized area. The dye or drugs remains localized based on the osmotic properties of semi-permeable membranes. Said osmotic properties have no effect on radiation.

The injection may also be performed through a standard wire localization needle. A localization wire is then advanced into the marker to further enhance localization.

The methylene blue, tolulene blue, or other medical-grade dye may also be mixed with a radioopaque contrast agent if a mammogram or X-rays will be used to localize a wire position. When using a mammogram to check the wire position, the contrast agent will facilitate the determination as to whether or not the marker has been found.

If a mammogram/X-ray is used to find the wire in a case where a radioopaque contrast agent but no medical dye was injected into the marker at the time the wire localization was performed, such medical-grade dye or therapeutic substance, or both, can be injected into the marker at that time. The marker is found under ultrasound first. The dye or therapeutic substance, or both, is then injected into the marker to make it more visible on mammogram, visual identification, and other imaging modalities.

A dye or therapeutic substance can also be injected into tissue in the immediate vicinity of the marker and osmotic diffusion will result in absorption of such dye or therapeutic substance by said marker upon contact.

Compounds having utility as marking agents include, for example, fludeoxyglucose (¹⁸F) or fluorodeoxyglucose (¹⁸F), commonly abbreviated ¹⁸F-FDG or FDG. This compound is a radiopharmaceutical that can be used in conjunction with fluorescent material in a form of nano particles, such as plasmonic fluorescent quantum dots, which are injected into the hydrogel to facilitate locating tumor cells under different wavelength light sources, such as UV light, during surgery.

These compounds may serve as imaging agents or marking agents, depending upon the molecule. The novel marker may also be injected with nanoparticles that could function as imaging or marking agents or nanoparticles that could yield a high dose local treatment of a tumor by continuously diffusing out of the hydrogel at a slow rate.

It will thus be seen that the objects set forth above, and those made apparent from the foregoing disclosure, are efficiently attained and since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

I/We claim:

1. A biopsy site marker comprising:
a body defining an outer perimeter of the marker, the body including hydrogel, the hydrogel being configured to transition from a dehydrated state to a hydrated state, the hydrogel being configured further to expand in size when transitioning from the dehydrated state to the hydrated state,
the hydrogel including a radiation emitting material, the radiation emitting material being configured to permit detection of the marker using a gamma camera.

2. The marker of claim 1, further comprising a localization element including a wire, a portion of the localization element being disposed within the body.

3. The marker of claim 2, the localization element being configured to detection under x-ray visualization.

4. The marker of claim 1, the hydrogel further including a plurality of nanoparticles, the nanoparticles being configured to emit electromagnetic radiation.

5. The marker of claim 1, the hydrogel further including a radiopaque contrast agent.

6. The marker of claim 1, the hydrogel further including a medical dye.

7. The marker of claim 6, the medical dye including one or more of methylene blue, toluene blue, gentian violet, methyl violet, dichlorophenolindophenol, fluorescein, Prussian blue, Egyptian blue, Han purple, potassium ferrocyanide, potassium ferricyanide, and phenothiazine.

8. The marker of claim 1, the hydrogel being configured to form a semi-permeable membrane.

9. A system for locating a biopsy site, the system comprising:
(a) a marker, the marker including a hydrogel body, the hydrogel body being configured to transition from a dehydrated state to a hydrated state, the hydrogel being configured further to expand in size when transitioning from the dehydrated state to the hydrated state, the hydrogel body including a radiation emitting compound; and
(b) a gamma camera, the gamma camera being configured to detect radiation emitted from the radiation emitting compound to locate the position of the marker within tissue.

10. The system of claim 9, further comprising a localization element including a wire, a portion of the localization element being disposed within the hydrogel body.

11. The marker of claim 10, the localization element being configured to detection under x-ray visualization.

12. The marker of claim 9, the hydrogel body further including a plurality of nanoparticles, the nanoparticles being configured to emit electromagnetic radiation.

13. The marker of claim 9, the hydrogel body further including a radiopaque contrast agent.

14. The marker of claim 9, the hydrogel body further including a medical dye.

15. The marker of claim 14, the medical dye including one or more of methylene blue, toluene blue, gentian violet, methyl violet, dichlorophenolindophenol, fluorescein, Prussian blue, Egyptian blue, Han purple, potassium ferrocyanide, potassium ferricyanide, and phenothiazine.

16. The marker of claim 9, the hydrogel being configured to form a semi-permeable membrane.
